(19) United States
(12) Patent Application Publication
(10) Pub. No.: US 2010/0130712 A1
(43) Pub. Date: May 27, 2010

(54) ADAMANTANE DERIVATIVE, RESIN COMPOSITION USING THE SAME, AND RESIN CURED PRODUCT

(75) Inventors: Katsuki Ito, Chiba (JP); Yasunari Okada, Chiba (JP); Hideki Yamane, Chiba (JP); Shinobu Yamao, Chiba (JP)

Correspondence Address: OBLON, SPIVAK, MCCLELLAND MAIER & NEUSTADT, L.L.P., 1940 DUKE STREET, ALEXANDRIA, VA, 22314, US

(73) Assignee: Idemitsu Kosan Co., Ltd., Chiyoda-ku (JP)

(21) Appl. No.: 12/594,254

(22) PCT Filed: Mar. 31, 2008
(86) PCT No.: PCT/JP08/56303

§ 371 Date: Oct. 1, 2009

(30) Foreign Application Priority Data
Apr. 3, 2007 (JP) 2007-097485

(51) Int. Cl. C08F 124/00; C07D 205/04

(52) U.S. Cl. 526/266; 548/950

(57) ABSTRACT

Provided is a cured resin of an adamantane derivative having a specific structure, which has excellent optical properties such as transparency and (long-term) light stability, long-term heat resistance, dielectric constant, and mechanical properties, and which can be utilized suitably in the field of electronic and optical materials.

### TECHNICAL FIELD

The present invention relates to a novel adamantane derivative and a method for producing the same, more particularly to an adamantane derivative suitable as a monomer for producing a resin composition and a cured resin which are useful in the field of electronic and optical materials, a method for producing the same, and a resin composition and a cured resin obtained by using the adamantane derivative.

### BACKGROUND ART

Adamantane has a cage-like structure obtained by condensation of four cyclohexane rings and is a highly symmetrical and stable compound. Its derivatives show unique functions and are known to be useful as raw materials for medicine and highly functional industrial materials. For example, attempts are being made to use adamantane for an optical disc substrate, optical fiber, lens, and the like because it possesses, for example, optical characteristic features and heat resistance (see Patent Documents 1 and 2).

Also, attempts are being made to use adamantane esters as raw materials of photoresist resins by making use of their acid sensitivity, dry etching resistance, transparency to ultraviolet light, and the like (see Patent Document 3).

Recently, in the field of electronic and optical materials, there are efforts being made to enhance the performance of optical and electronic components including, specifically, improvement of flat panel displays based on liquid crystals and organic electroluminescence (organic EL) in terms of high-definition imaging, wide viewing angle, and high image quality; development of a light source using optical semiconductors, which exhibits high brightness, shorter wavelength, and white light emission; development of an electronic circuit, which operates at high frequency; and development of a circuit, communication, and the like based on the use of light.

With this enhanced performance of the optical and electronic components, the resins used in the coating material, encapsulation material, and adhesive for the optical and electronic components have also come to be expected to exhibit high performance. Thus, various heat-curable resins, light-curable resins, or thermoplastic resins are being applied according to characteristics thereof such as heat resistance, transparency solubility, adhesiveness, and the like. However, requirements for the performance of optical and electronic components and the like are increasing, and, improvement in performance has also been desired of the resins used for these.

For example, in an electronic circuit board where semiconductor devices and the like are integrated, increase in information volume and communication speed, and miniaturization of devices are progressing to make miniaturization, integration, and high-frequency operability of the circuit necessary. Further, optical circuits using optical waveguides and the like, which make higher-speed processing possible, are being investigated. When conventionally used epoxy and acrylic resins and the like are used as the encapsulation resins, adhesive resins, films, or lens resins for the electronic circuits or optical circuits, in an electronic circuit application, there are problems such as a high dielectric constant and insufficient heat resistance, and, in optical waveguide and LED encapsulation applications, there are problems such as lowering of transparency and yellowing due to deterioration of the resin.

The cured resin used in the aforementioned applications is required to exhibit performance to solve the above-mentioned problems. On the other hand, a monomer for producing the cured resin is required to have excellent storage stability and be usable in numerous ways in accordance with the intended use of the cured resin (that is, to be applicable under various curing conditions and to have a wide range of wide applications). However, there has not heretofore been available a monomer for producing a cured resin which can provide a cured resin having excellent transparency, heat resistance, solvent resistance, and the like, and which has good storage stability and is usable in numerous ways.

Patent Document 1: Japanese Patent Laid-Open Publication No. H6-305044
Patent Document 2: Japanese Patent Laid-Open Publication No. H9-302077

### DISCLOSURE OF THE INVENTION

### Problems to be Solved by the Invention

The present invention has been made under these circumstances and has objects (1) to provide a cured resin which has excellent optical properties such as transparency and (long-term) light resistance, long-term heat resistance, dielectric constant, and mechanical properties, and which can be utilized suitably in the field of electronic and optical materials, and (2) to provide an adamantane derivative which can be used for the production of the cured resin and which can be applied to various ways of use depending on the intended use of the cured resin.

### Means for Solving the Problems

The present inventors conducted diligent research to accomplish the aforementioned objects and, as a result, have found that an adamantane derivative having an adamantane skeleton and, further, an acrylate portion and a specific cyclic ether portion can suit the objects. This finding led to completion of the present invention.

That is, the present invention provides the following (1) to (8):

(1) An adamantane derivative having a structure represented by the general formula (I):

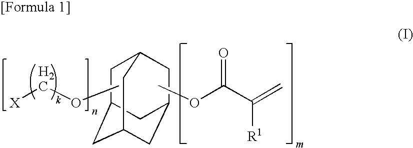

wherein, R¹ represents a hydrogen atom, an alkyl group having 1 to 4 carbon atoms, or a trifluoromethyl group; X represents a cyclic ether group which can undergo cationic ring-opening polymerization; k represents an integer of from 0 to 7; m and n each independently represent an integer of 1 or larger, and m+n≦4.
(2) The adamantane derivative according to the above (1), wherein X in the general formula (I) is a group represented by the general formula (II) or (III):

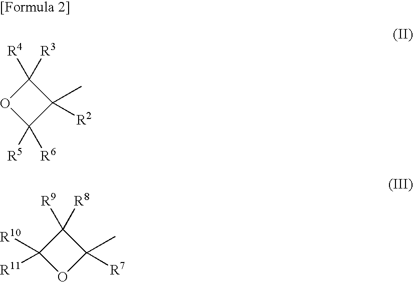

wherein, R² to R¹¹ each independently represent a hydrogen atom, a fluorine atom, an alkyl group having 1 to 4 carbon atoms, a phenyl group, or a perfluoroalkyl group having 1 to 4 carbon atoms.
(3) The adamantane derivative according to the above (1), wherein X in the general formula (I) is a group represented by the general formula (IV):

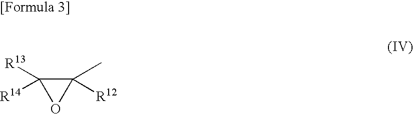

wherein, R¹² to R¹⁴ each independently represent a hydrogen atom, a fluorine atom, an alkyl group having 1 to 4 carbon atoms, a phenyl group, or a perfluoroalkyl group having 1 to 4 carbon atoms.
(4) An adamantane derivative represented by the general formula (V) or (VI)

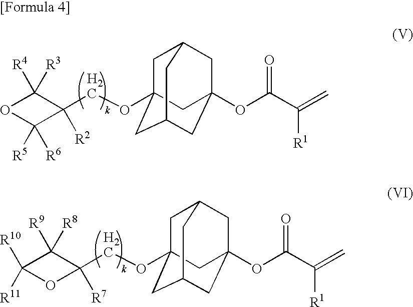

wherein, R¹ represents a hydrogen atom, an alkyl group having 1 to 4 carbon atoms, or a trifluoromethyl group; R² to R¹¹ each independently represent a hydrogen atom, a fluorine atom, an alkyl group having 1 to 4 carbon atoms, a phenyl group, or a perfluoroalkyl group having 1 to 4 carbon atoms; k represents an integer of from 0 to 7.
(5) An adamantane derivative represented by the general formula (VII):

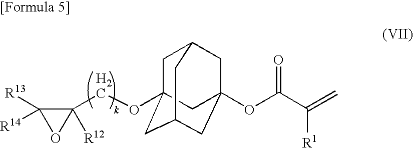

wherein, R¹ represents a hydrogen atom, an alkyl group having 1 to 4 carbon atoms, or a trifluoromethyl group; R¹² to R¹⁴ each independently represent a hydrogen atom, a fluorine atom, an alkyl group having 1 to 4 carbon atoms, a phenyl group, or a perfluoroalkyl group having 1 to 4 carbon atoms; k represents an integer of from 0 to 7.
(6) A method for producing the adamantane derivative according to the above (2), wherein oxetanyl alcohol and an adamantane derivative represented by the general formula (VIII) are reacted.

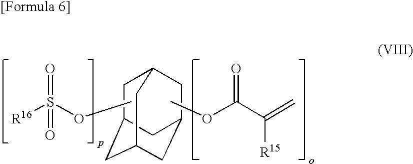

wherein, R¹⁵ represents a hydrogen atom, an alkyl group having 1 to 4 carbon atoms, or a trifluoromethyl group; R¹⁶ represents an alkyl group having 1 to 3 carbon atoms; o and p each independently represent an integer of 1 or larger, and o+p≦4.
(7) A method for producing the adamantane derivative according to the above (3), wherein a halogen-containing alcohol and an adamantane derivative represented by the general formula (VIII) are reacted.
(8) A cured resin obtained by polymerizing the adamantane derivative represented by the general formula (I) by a cationic polymerization initiator and/or a radical polymerization initiator.

### EFFECTS OF THE INVENTION

According to the present invention, (1) there is provided a cured resin which has excellent optical properties such as transparency and (long-term) light stability, long-term heat resistance, dielectric constant, and mechanical properties, and which can be utilized suitably in the field of electronic and optical materials, and, further, (2) there is provided an adamantane derivative which can be utilized for the production of the cured resin and which can be applied to numerous ways of use depending on the intended use of the cured resin.

### BEST MODE FOR CARRYING OUT THE INVENTION

The adamantane derivative of the present invention is a compound represented by the general formula (I):

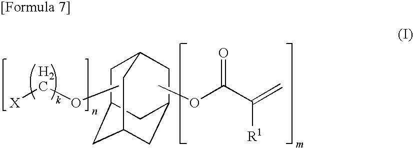

In the general formula (I), R¹ represents a hydrogen atom, an alkyl group having 1 to 4 carbon atoms, or a trifluoromethyl group; X represents a cyclic ether group which can undergo cationic ring-opening polymerization; k represents an integer of from 0 to 7; m and n each independently represent an integer of 1 or larger, and m+n≦4.

The adamantane derivative of the present invention comprises an acrylate portion and a cyclic ether portion as described above, Therefore, in producing a cured resin, there can be utilized a radical polymerization reaction and a cationic polymerization reaction. Or, it is possible to use these together and, thus, in accordance with the intended use of the cured resin, the adamantane derivative can be used under various curing conditions. Meanwhile, in the present specification, the acrylate portion refers to a portion represented by

“H₂C═C(R¹)C(O)O—”

wherein R¹ represents a hydrogen atom, an alkyl group having 1 to 4 carbon atoms, or a trifluoromethyl group.

In the general formula (I), specific examples of an alkyl group having 1 to 4 carbon atoms include a methyl group, an ethyl group, various propyl groups, and various butyl groups.

In the general formula (I), specific examples of X include oxetanyl groups represented by the general formula (II) and (III):

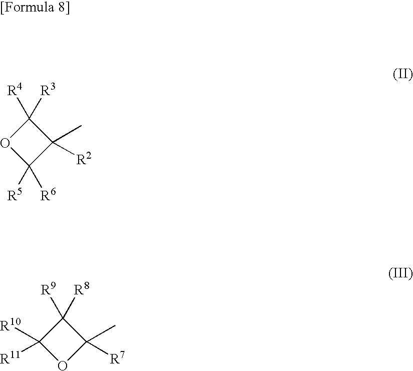

wherein, R² to R¹¹ each independently represent a hydrogen atom, a fluorine atom, an alkyl group having 1 to 4 carbon atoms, a phenyl group, or a perfluoro alkyl group having 1 to 4 carbon atoms;
and an oxiranyl group represented by the general formula (IV):

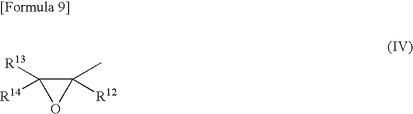

wherein, R¹² to R¹¹ each independently represent a hydrogen atom, a fluorine atom, an alkyl group having 1 to 4 carbon atoms, a phenyl group, or a perfluoro alkyl group having 1 to 4 carbon atoms.

Specific examples of the compounds represented by the general formula (I) include the following compounds. Meanwhile, in the present specification, (meth)acrylate represents acrylate or methacrylate.
3-[(3-methyloxetan-3-yl)methoxy]-1-adamantyl(meth)acrylate,
3-[(3-ethyloxetan-3-yl)methoxy]-1-adamantyl(meth)acrylate,
3-[(3-propyloxetan-3-yl)methoxy]-1-adamantyl(meth)acrylate,
3-[(3-butyloxetan-3-yl)methoxy]-1-adamantyl(meth)acrylate,
3-[(3-methyloxetan-3-yl)ethoxy]-1-adamantyl(meth)acrylate,
3-[(3-ethyloxetan-3-yl)ethoxy]-1-adamantyl(meth)acrylate,
3-[(3-propyloxetan-3-yl)ethoxy]-1-adamantyl(meth)acrylate,
3-[(3-butyloxetan-3-yl)ethoxy]-1-adamantyl(meth)acrylate,
3-[(3-methyloxetan-3-yl)propoxy]-1-adamantyl(meth)acrylate,
3-[(3-ethyloxetan-3-yl)propoxy]-1-adamantyl(meth)acrylate,
3-[(3-propyloxetan-3-yl)propoxy]-1-adamantyl(meth)acrylate,
3-[(3-butyloxetan-3-yl)propoxy)]-1-adamantyl(meth)acrylate,
3-[(3-methyloxetan-3-yl)butoxy]-1-adamantyl(meth)acrylate,
3-[(3-ethyloxetan-3-yl)butoxy]-1-adamantyl(meth)acrylate,
3-[(3-propyloxetan-3-yl)butoxy]-1-adamantyl(meth)acrylate,
3-[(3-butyloxetan-3-yl)butoxy]-1-adamantyl(meth)acrylate,
3-[(3-methyloxetan-3-yl)pentoxy]-1-adamantyl(meth)acrylate,
3-[(3-ethyloxetan-3-yl)pentoxy]-1-adamantyl(meth)acrylate,
3-[(3-propyloxetan-3-yl)pentoxy]-1-adamantyl(meth)acrylate,
3-[(3-butyloxetan-3-yl)pentoxy]-1-adamantyl(meth)acrylate,
3-[(3-methyloxetan-3-yl)hexoxy]-1-adamantyl(meth)acrylate,
3-[(3-ethyloxetan-3-yl)hexoxy]-1-adamantyl(meth)acrylate,
3-[(3-propyloxetan-3-yl)hexoxy]-1-adamantyl(meth)acrylate,
3-[(3-butyloxetan-3-yl)hexoxy]-1-adamantyl(meth)acrylate,
3-[(3-methyloxetan-3-yl)heptoxy]-1-adamantyl(meth)acrylate,
3-[(3-ethyloxetan-3-yl)heptoxy]-1-adamantyl(meth)acrylate,
3-[(3-propyloxetan-3-yl)heptoxy]-1-adamantyl(meth)acrylate,
3-[(3-butyloxetan-3-yl)heptoxy]-1-adamantyl(meth)acrylate,
3-[(2-methyloxetan-2-yl)methoxy]-1-adamantyl(meth)acrylate,
3-[(2-ethyloxetan-2-yl)methoxy]-1-adamantyl(meth)acrylate,
3-[(2-propyloxetan-2-yl)methoxy]-1-adamantyl(meth)acrylate,
3-[(2-butyloxetan-2-yl)methoxy]-1-adamantyl(meth)acrylate,
3-[(2-methyloxetan-2-yl)ethoxy]-1-adamantyl(meth)acrylate,
3-[(2-ethyloxetan-2-yl)ethoxy]-1-adamantyl(meth)acrylate,
3-[(2-propyloxetan-2-yl)ethoxy]-1-adamantyl(meth)acrylate,
3-[(2-butyloxetan-2-yl)ethoxy]-1-adamantyl(meth)acrylate,
3-[(2-methyloxetan-2-yl)propoxy]-1-adamantyl(meth)acrylate,
3-[(2-ethyloxetan-2-yl)propoxy]-1-adamantyl(meth)acrylate,
3-[(2-propyloxetan-2-yl)propoxy]-1-adamantyl(meth)acrylate,
3-[(2-butyloxetan-2-yl)propoxy]-1-adamantyl(meth)acrylate,
3-[(2-methyloxetan-2-yl)butoxy]-1-adamantyl(meth)acrylate,
3-[(2-ethyloxetan-2-yl)butoxy]-1-adamantyl(meth)acrylate,
3-[(2-propyloxetan-2-yl)butoxy]-1-adamantyl(meth)acrylate,
3-[(2-butyloxetan-2-yl)butoxy]-1-adamantyl(meth)acrylate,
3-[(2-methyloxetan-2-yl)pentoxy]-1-adamantyl(meth)acrylate,
3-[(2-ethyloxetan-2-yl)pentoxy]-1-adamantyl(meth)acrylate,
3-[(2-propyloxetan-2-yl)pentoxy]-1-adamantyl(meth)acrylate,
3-[(2-butyloxetan-2-yl)pentoxy]-1-adamantyl(meth)acrylate,
3-[(2-methyloxetan-2-yl)hexoxy]-1-adamantyl(meth)acrylate,
3-[(2-ethyloxetan-2-yl)hexoxy]-1-adamantyl(meth)acrylate,
3-[(2-propyloxetan-2-yl)hexoxy]-1-adamantyl(meth)acrylate,
3-[(2-butyloxetan-2-yl)hexoxy]-1-adamantyl(meth)acrylate,
3-[(2-methyloxetan-2-yl)heptoxy]-1-adamantyl(meth)acrylate,
3-[(2-ethyloxetan-2-yl)heptoxy]-1-adamantyl(meth)acrylate,
3-[(2-propyloxetan-2-yl)heptoxy]-1-adamantyl(meth)acrylate,
3-[(2-butyloxetan-2-yl)heptoxy]-1-adamantyl(meth)acrylate,
3,5-bis[(3-methyloxetan-3-yl)methoxy]-1-adamantyl(meth)acrylate,
3,5-bis[(3-ethyloxetan-3-yl)methoxy]-1-adamantyl(meth)acrylate,
3,5-bis[(3-propyloxetan-3-yl)methoxy]-1-adamantyl(meth)acrylate,
3,5-bis[(3-butyloxetan-3-yl)methoxy]-1-adamantyl(meth)acrylate,
3,5-bis[(3-methyloxetan-3-yl)ethoxy]-1-adamantyl(meth)acrylate,
3,5-bis[(3-ethyloxetan-3-yl)ethoxy]-1-adamantyl(meth)acrylate,
3,5-bis[(3-propyloxetan-3-yl)ethoxy]-1-adamantyl(meth)acrylate,
3,5-bis[(3-butyloxetan-3-yl)ethoxy]-1-adamantyl(meth)acrylate,
3,5-bis[(3-methyloxetan-3-yl)propoxy]-1-adamantyl(meth)acrylate,
3,5-bis[(3-ethyloxetan-3-yl)propoxy]-1-adamantyl(meth)acrylate,
3,5-bis[(3-propyloxetan-3-yl)propoxy]-1-adamantyl(meth)acrylate,
3,5-bis[(3-butyloxetan-3-yl)propoxy]-1-adamantyl(meth)acrylate,
3,5-bis[(3-methyloxetan-3-yl)butoxy]-1-adamantyl(meth)acrylate,
3,5-bis[(3-ethyloxetan-3-yl)butoxy]-1-adamantyl(meth)acrylate,
3,5-bis[(3-propyloxetan-3-yl)butoxy]-1-adamantyl(meth)acrylate,
3,5-bis[(3-butyloxetan-3-yl)butoxy]-1-adamantyl(meth)acrylate,
3,5-bis[(3-methyloxetan-3-yl)pentoxy]-1-adamantyl(meth)acrylate,
3,5-bis[(3-ethyloxetan-3-yl)pentoxy]-1-adamantyl(meth)acrylate,
3,5-bis[(3-propyloxetan-3-yl)pentoxy]-1-adamantyl(meth)acrylate,
3,5-bis[(3-butyloxetan-3-yl)pentoxy]-1-adamantyl(meth)acrylate,
3,5-bis[(3-methyloxetan-3-yl)hexoxy]-1-adamantyl(meth)acrylate,
3,5-bis[(3-ethyloxetan-3-yl)hexoxy]-1-adamantyl(meth)acrylate,
3,5-bis[(3-propyloxetan-3-yl)hexoxy]-1-adamantyl(meth)acrylate,
3,5-bis[(3-butyloxetan-3-yl)hexoxy]-1-adamantyl(meth)acrylate,
3,5-bis[(3-methyloxetan-3-yl)heptoxy]-1-adamantyl(meth)acrylate,
3,5-bis[(3-ethyloxetan-3-yl)heptoxy]-1-adamantyl(meth)acrylate,
3,5-bis[(3-propyloxetan-3-yl)heptoxy]-1-adamantyl(meth)acrylate,
3,5-bis[(3-butyloxetan-3-yl)heptoxy]-1-adamantyl(meth)acrylate,
3,5-bis[(2-methyloxetan-2-yl)methoxy]-1-adamantyl(meth)acrylate,
3,5-bis[(2-ethyloxetan-2-yl)methoxy]-1-adamantyl(meth)acrylate,
3,5-bis[(2-propyloxetan-2-yl)methoxy]-1-adamantyl(meth)acrylate,
3,5-bis[(2-butyloxetan-2-yl)methoxy]-1-adamantyl(meth)acrylate,
3,5-bis[(2-methyloxetan-2-yl)ethoxy]-1-adamantyl(meth)acrylate,
3,5-bis[(2-ethyloxetan-2-yl)ethoxy]-1-adamantyl(meth)acrylate,
3,5-bis[(2-propyloxetan-2-yl)ethoxy]-1-adamantyl(meth)acrylate,
3,5-bis[(2-butyloxetan-2-yl)ethoxy]-1-adamantyl(meth)acrylate,
3,5-bis[(2-methyloxetan-2-yl)propoxy]-1-adamantyl(meth)acrylate,
3,5-bis[(2-ethyloxetan-2-yl)propoxy]-1-adamantyl(meth)acrylate,
3,5-bis[(2-propyloxetan-2-yl)propoxy]-1-adamantyl(meth)acrylate,
3,5-bis[(2-butyloxetan-2-yl)propoxy]-1-adamantyl(meth)acrylate,
3,5-bis[(2-methyloxetan-2-yl)butoxy]-1-adamantyl(meth)acrylate,
3,5-bis[(2-ethyloxetan-2-yl)butoxy]-1-adamantyl(meth)acrylate,
3,5-bis[(2-propyloxetan-2-yl)butoxy]-1-adamantyl(meth)acrylate,
3,5-bis[(2-butyloxetan-2-yl)butoxy]-1-adamantyl(meth)acrylate,
3,5-bis[(2-methyloxetan-2-yl)pentoxy]-1-adamantyl(meth)acrylate,
3,5-bis[(2-ethyloxetan-2-yl)pentoxy]-1-adamantyl(meth)acrylate,
3,5-bis[(2-propyloxetan-2-yl)pentoxy]-1-adamantyl(meth)acrylate,
3,5-bis[(2-butyloxetan-2-yl)pentoxy]-1-adamantyl(meth)acrylate,
3,5-bis[(2-methyloxetan-2-yl)hexoxy]-1-adamantyl(meth)acrylate,
3,5-bis[(2-ethyloxetan-2-yl)hexoxy]-1-adamantyl(meth)acrylate,
3,5-bis[(2-propyloxetan-2-yl)hexoxy]-1-adamantyl(meth)acrylate,
3,5-bis[(2-butyloxetan-2-yl)hexoxy]-1-adamantyl(meth)acrylate,
3,5-bis[(2-methyloxetan-2-yl)heptoxy]-1-adamantyl(meth)acrylate,
3,5-bis[(2-ethyloxetan-2-yl)heptoxy]-1-adamantyl(meth)acrylate,
3,5-bis[(2-propyloxetan-2-yl)heptoxy]-1-adamantyl(meth)acrylate,
3,5-bis[(2-butyloxetan-2-yl)heptoxy]-1-adamantyl(meth)acrylate,
3,5,7-tris[(3-methyloxetan-3-yl)methoxy]-1-adamantyl(meth)acrylate,
3,5,7-tris[(3-ethyloxetan-3-yl)methoxy]-1-adamantyl(meth)acrylate,
3,5,7-tris[(3-propyloxetan-3-yl)methoxy]-1-adamantyl(meth)acrylate,
3,5,7-tris[(3-butyloxetan-3-yl)methoxy]-1-adamantyl(meth)acrylate,
3,5,7-tris[(3-methyloxetan-3-yl)ethoxy]-1-adamantyl(meth)acrylate,
3,5,7-tris[(3-ethyloxetan-3-yl)ethoxy]-1-adamantyl(meth)acrylate,
3,5,7-tris[(3-propyloxetan-3-yl)ethoxy]-1-adamantyl(meth)acrylate,
3,5,7-tris[(3-butyloxetan-3-yl)ethoxy]-1-adamantyl(meth)acrylate,
3,5,7-tris[(3-methyloxetan-3-yl)propoxy]-1-adamantyl(meth)acrylate,
3,5,7-tris[(3-ethyloxetan-3-yl)propoxy]-1-adamantyl(meth)acrylate,
3,5,7-tris[(3-propyloxetan-3-yl)propoxy]-1-adamantyl(meth)acrylate,
3,5,7-tris[(3-butyloxetan-3-yl)propoxy]-1-adamantyl(meth)acrylate,
3,5,7-tris[(3-methyloxetan-3-yl)butoxy]-1-adamantyl(meth)acrylate,
3,5,7-tris[(3-ethyloxetan-3-yl)butoxy]-1-adamantyl(meth)acrylate,
3,5,7-tris[(3-propyloxetan-3-yl)butoxy]-1-adamantyl(meth)acrylate,
3,5,7-tris[(3-butyloxetan-3-yl)butoxy]-1-adamantyl(meth)acrylate,
3,5,7-tris[(3-methyloxetan-3-yl)pentoxy]-1-adamantyl(meth)acrylate,
3,5,7-tris[(3-ethyloxetan-3-yl)pentoxy]-1-adamantyl(meth)acrylate,
3,5,7-tris[(3-propyloxetan-3-yl)pentoxy]-1-adamantyl(meth)acrylate,
3,5,7-tris[(3-butyloxetan-3-yl)pentoxy]-1-adamantyl(meth)acrylate,
3,5,7-tris[(3-methyloxetan-3-yl)hexoxy]-1-adamantyl(meth)acrylate,
3,5,7-tris[(3-ethyloxetan-3-yl)hexoxy]-1-adamantyl(meth)acrylate,
3,5,7-tris[(3-propyloxetan-3-yl)hexoxy]-1-adamantyl(meth)acrylate,
3,5,7-tris[(3-butyloxetan-3-yl)hexoxy]-1-adamantyl(meth)acrylate,
3,5,7-tris[(3-methyloxetan-3-yl)heptoxy]-1-adamantyl(meth)acrylate,
3,5,7-tris[(3-ethyloxetan-3-yl)heptoxy]-1-adamantyl(meth)acrylate,
3,5,7-tris[(3-propyloxetan-3-yl)heptoxy]-1-adamantyl(meth)acrylate,
3,5,7-tris[(3-butyloxetan-3-yl)heptoxy]-1-adamantyl(meth)acrylate,
3,5,7-tris[(2-methyloxetan-2-yl)methoxy]-1-adamantyl(meth)acrylate,
3,5,7-tris[(2-ethyloxetan-2-yl)methoxy]-1-adamantyl(meth)acrylate,
3,5,7-tris[(2-propyloxetan-2-yl)methoxy]-1-adamantyl(meth)acrylate,
3,5,7-tris[(2-butyloxetan-2-yl)methoxy]-1-adamantyl(meth)acrylate,
3,5,7-tris[(2-methyloxetan-2-yl)ethoxy]-1-adamantyl(meth)acrylate,
3,5,7-tris[(2-ethyloxetan-2-yl)ethoxy]-1-adamantyl(meth)acrylate,
3,5,7-tris[(2-propyloxetan-2-yl)ethoxy]-1-adamantyl(meth)acrylate,
3,5,7-tris[(2-butyloxetan-2-yl)ethoxy]-1-adamantyl(meth)acrylate,
3,5,7-tris[(2-methyloxetan-2-yl)propoxy]-1-adamantyl(meth)acrylate,
3,5,7-tris[(2-ethyloxetan-2-yl)propoxy]-1-adamantyl(meth)acrylate,
3,5,7-tris[(2-propyloxetan-2-yl)propoxy]-1-adamantyl(meth)acrylate,
3,5,7-tris[(2-butyloxetan-2-yl)propoxy]-1-adamantyl(meth)acrylate,
3,5,7-tris[(2-methyloxetan-2-yl)butoxy]-1-adamantyl(meth)acrylate,
3,5,7-tris[(2-ethyloxetan-2-yl)butoxy]-1-adamantyl(meth)acrylate,
3,5,7-tris[(2-propyloxetan-2-yl)butoxy]-1-adamantyl(meth)acrylate,
3,5,7-tris[(2-butyloxetan-2-yl)butoxy]-1-adamantyl(meth)acrylate,
3,5,7-tris[(2-methyloxetan-2-yl)pentoxy]-1-adamantyl(meth)acrylate,
3,5,7-tris[(2-ethyloxetan-2-yl)pentoxy]-1-adamantyl(meth)acrylate,
3,5,7-tris[(2-propyloxetan-2-yl)pentoxy]-1-adamantyl(meth)acrylate,
3,5,7-tris[(2-butyloxetan-2-yl)pentoxy]-1-adamantyl(meth)acrylate,
3,5,7-tris[(2-methyloxetan-2-yl)hexoxy]-1-adamantyl(meth)acrylate,
3,5,7-tris[(2-ethyloxetan-2-yl)hexoxy]-1-adamantyl(meth)acrylate,
3,5,7-tris[(2-propyloxetan-2-yl)hexoxy]-1-adamantyl(meth)acrylate,
3,5,7-tris[(2-butyloxetan-2-yl)hexoxy]-1-adamantyl(meth)acrylate,
3,5,7-tris[(2-methyloxetan-2-yl)heptoxy]-1-adamantyl(meth)acrylate,
3,5,7-tris[(2-ethyloxetan-2-yl)heptoxy]-1-adamantyl(meth)acrylate,
3,5,7-tris[(2-propyloxetan-2-yl)heptoxy]-1-adamantyl(meth)acrylate,
3,5,7-tris[(2-butyloxetan-2-yl)heptoxy]-1-adamantyl(meth)acrylate,
5-[(3-methyloxetan-3-yl)methoxy]-1,3-adamantyl di(meth)acrylate,
5-[(3-ethyloxetan-3-yl)methoxy]-1,3-adamantyl di(meth)acrylate,
5-[(3-propyloxetan-3-yl)methoxy]-1,3-adamantyl di(meth)acrylate,
5-[(3-butyloxetan-3-yl)methoxy]-1,3-adamantyl di(meth)acrylate,
5-[(3-methyloxetan-3-yl)ethoxy]-1,3-adamantyl di(meth)acrylate,
5-[(3-ethyloxetan-3-yl)ethoxy]-1,3-adamantyl di(meth)acrylate,
5-[(3-propyloxetan-3-yl)ethoxy]-1,3-adamantyl di(meth)acrylate,
5-[(3-butyloxetan-3-yl)ethoxy]-1,3-adamantyl di(meth)acrylate,
5-[(3-methyloxetan-3-yl)propoxy]-1,3-adamantyl di(meth)acrylate,
5-[(3-ethyloxetan-3-yl)propoxy]-1,3-adamantyl di(meth)acrylate,
5-[(3-propyloxetan-3-yl)propoxy]-1,3-adamantyl di(meth)acrylate,
5-[(3-butyloxetan-3-yl)propoxy]-1,3-adamantyl di(meth)acrylate,
5-[(3-methyloxetan-3-yl)butoxy]-1,3-adamantyl di(meth)acrylate,
5-[(3-ethyloxetan-3-yl)butoxy]-1,3-adamantyl di(meth)acrylate,
5-[(3-propyloxetan-3-yl)butoxy]-1,3-adamantyl di(meth)acrylate,
5-[(3-butyloxetan-3-yl)butoxy]-1,3-adamantyl di(meth)acrylate,
5-[(3-methyloxetan-3-yl)pentoxy]-1,3-adamantyl di(meth)acrylate,
5-[(3-ethyloxetan-3-yl)pentoxy]-1,3-adamantyl di(meth)acrylate,
5-[(3-propyloxetan-3-yl)pentoxy]-1,3-adamantyl di(meth)acrylate,
5-[(3-butyloxetan-3-yl)pentoxy]-1,3-adamantyl di(meth)acrylate,
5-[(3-methyloxetan-3-yl)hexoxy]-1,3-adamantyl di(meth)acrylate,
5-[(3-ethyloxetan-3-yl)hexoxy]-1,3-adamantyl di(meth)acrylate,
5-[(3-propyloxetan-3-yl)hexoxy]-1,3-adamantyl di(meth)acrylate,
5-[(3-butyloxetan-3-yl)hexoxy]-1,3-adamantyl di(meth)acrylate,
5-[(3-methyloxetan-3-yl)heptoxy]-1,3-adamantyl di(meth)acrylate,
5-[(3-ethyloxetan-3-yl)heptoxy]-1,3-adamantyl di(meth)acrylate,
5-[(3-propyloxetan-3-yl)heptoxy]-1,3-adamantyl di(meth)acrylate,
5-[(3-butyloxetan-3-yl)heptoxy]-1,3-adamantyl di(meth)acrylate,
5-[(2-methyloxetan-2-yl)methoxy]-1,3-adamantyl di(meth)acrylate,
5-[(2-ethyloxetan-2-yl)methoxy]-1,3-adamantyl di(meth)acrylate,
5-[(2-propyloxetan-2-yl)methoxy]-1,3-adamantyl di(meth)acrylate,
5-[(2-butyloxetan-2-yl)methoxy]-1,3-adamantyl di(meth)acrylate,
5-[(2-methyloxetan-2-yl)ethoxy]-1,3-adamantyl di(meth)acrylate,
5-[(2-ethyloxetan-2-yl)ethoxy]-1,3-adamantyl di(meth)acrylate,
5-[(2-propyloxetan-2-yl)ethoxy]-1,3-adamantyl di(meth)acrylate,
5-[(2-butyloxetan-2-yl)ethoxy]-1,3-adamantyl di(meth)acrylate,
5-[(2-methyloxetan-2-yl)propoxy]-1,3-adamantyl di(meth)acrylate,
5-[(2-ethyloxetan-2-yl)propoxy]-1,3-adamantyl di(meth)acrylate,
5-[(2-propyloxetan-2-yl)propoxy]-1,3-adamantyl di(meth)acrylate,
5-[(2-butyloxetan-2-yl)propoxy]-1,3-adamantyl di(meth)acrylate,
5-[(2-methyloxetan-2-yl)butoxy]-1,3-adamantyl di(meth)acrylate,
5-[(2-ethyloxetan-2-yl)butoxy]-1,3-adamantyl di(meth)acrylate,
3-[(2-propyloxetan-2-yl)butoxy]-1,3-adamantyl di(meth)acrylate,
5-[(2-butyloxetan-2-yl)butoxy]-1,3-adamantyl di(meth)acrylate,
5-[(2-methyloxetan-2-yl)pentoxy]-1,3-adamantyl di(meth)acrylate,
5-[(2-ethyloxetan-2-yl)pentoxy]-1,3-adamantyl di(meth)acrylate,
5-[(2-propyloxetan-2-yl)pentoxy]-1,3-adamantyl di(meth)acrylate,
5-[(2-butyloxetan-2-yl)pentoxy]-1,3-adamantyl di(meth)acrylate,
5-[(2-methyloxetan-2-yl)hexoxy]-1,3-adamantyl di(meth)acrylate,
5-[(2-ethyloxetan-2-yl)hexoxy]-1,3-adamantyl di(meth)acrylate,
5-[(2-propyloxetan-2-yl)hexoxy]-1,3-adamantyl di(meth)acrylate,
5-[(2-butyloxetan-2-yl)hexoxy]-1,3-adamantyl di(meth)acrylate,
5-[(2-methyloxetan-2-yl)heptoxy]-1,3-adamantyl di(meth)acrylate,
5-[(2-ethyloxetan-2-yl)heptoxy]-1,3-adamantyl di(meth)acrylate,
5-[(2-propyloxetan-2-yl)heptoxy]-1,3-adamantyl di(meth)acrylate,
5-[(2-butyloxetan-2-yl)heptoxy]-1,3-adamantyl di(meth)acrylate,
5,7-bis[(3-methyloxetan-3-yl)methoxy]-1,3-adamantyl di(meth)acrylate,
5,7-bis[(3-ethyloxetan-3-yl)methoxy]-1,3-adamantyl di(meth)acrylate,
5,7-bis[(3-propyloxetan-3-yl)methoxy]-1,3-adamantyl di(meth)acrylate,
5,7-bis[(3-butyloxetan-3-yl)methoxy]-1,3-adamantyl di(meth)acrylate,
5,7-bis[(3-methyloxetan-3-yl)ethoxy]-1,3-adamantyl di(meth)acrylate,
5,7-bis[(3-ethyloxetan-3-yl)ethoxy]-1,3-adamantyl di(meth)acrylate,
5,7-bis[(3-propyloxetan-3-yl)ethoxy]-1,3-adamantyl di(meth)acrylate,
5,7-bis[(3-butyloxetan-3-yl)ethoxy]-1,3-adamantyl di(meth)acrylate,
5,7-bis[(3-methyloxetan-3-yl)propoxy]-1,3-adamantyl di(meth)acrylate,
5,7-bis[(3-ethyloxetan-3-yl)propoxy]-1,3-adamantyl di(meth)acrylate,
5,7-bis[(3-propyloxetan-3-yl)propoxy]-1,3-adamantyl di(meth)acrylate,
5,7-bis[(3-butyloxetan-3-yl)propoxy]-1,3-adamantyl di(meth)acrylate,
5-[(3-methyloxetan-3-yl)butoxy]-1,3-adamantyl di(meth)acrylate,
5,7-bis[(3-ethyloxetan-3-yl)butoxy]-1,3-adamantyl di(meth)acrylate,
5,7-bis[(3-propyloxetan-3-yl)butoxy]-1,3-adamantyl di(meth)acrylate,
5,7-bis[(3-butyloxetan-3-yl)butoxy]-1,3-adamantyl di(meth)acrylate,
5,7-bis[(3-methyloxetan-3-yl)pentoxy]-1,3-adamantyl di(meth)acrylate,
5,7-bis[(3-ethyloxetan-3-yl)pentoxy]-1,3-adamantyl di(meth)acrylate,
5,7-bis[(3-propyloxetan-3-yl)pentoxy]-1,3-adamantyl di(meth)acrylate,
5,7-bis[(3-butyloxetan-3-yl)pentoxy]-1,3-adamantyl di(meth)acrylate,
5,7-bis[(3-methyloxetan-3-yl)hexoxy]-1,3-adamantyl di(meth)acrylate,
5,7-bis[(3-ethyloxetan-3-yl)hexoxy]-1,3-adamantyl di(meth)acrylate,
5,7-bis[(3-propyloxetan-3-yl)hexoxy]-1,3-adamantyl di(meth)acrylate,
5,7-bis[(3-butyloxetan-3-yl)hexoxy]-1,3-adamantyl di(meth)acrylate,
5,7-bis[(3-methyloxetan-3-yl)heptoxy]-1,3-adamantyl di(meth)acrylate,
5,7-bis[(3-ethyloxetan-3-yl)heptoxy]-1,3-adamantyl di(meth)acrylate,
5,7-bis[(3-propyloxetan-3-yl)heptoxy]-1,3-adamantyl di(meth)acrylate,
5,7-bis[(3-butyloxetan-3-yl)heptoxy]-1,3-adamantyl di(meth)acrylate,
5,7-bis[(2-methyloxetan-2-yl)methoxy]-1,3-adamantyl di(meth)acrylate,
5,7-bis[(2-ethyloxetan-2-yl)methoxy]-1,3-adamantyl di(meth)acrylate,
5,7-bis[(2-propyloxetan-2-yl)methoxy]-1,3-adamantyl di(meth)acrylate,
5,7-bis[(2-butyloxetan-2-yl)methoxy]-1,3-adamantyl di(meth)acrylate,
5,7-bis[(2-methyloxetan-2-yl)ethoxy]-1,3-adamantyl di(meth)acrylate,
5,7-bis[(2-ethyloxetan-2-yl)ethoxy]-1,3-adamantyl di(meth)acrylate,
5,7-bis[(2-propyloxetan-2-yl)ethoxy]-1,3-adamantyl di(meth)acrylate,
5,7-bis[(2-butyloxetan-2-yl)ethoxy]-1,3-adamantyl di(meth)acrylate,
5,7-bis[(2-methyloxetan-2-yl)propoxy]-1,3-adamantyl di(meth)acrylate,
5,7-bis[(2-ethyloxetan-2-yl)propoxy]-1,3-adamantyl di(meth)acrylate,
5,7-bis[(2-propyloxetan-2-yl)propoxy]-1,3-adamantyl di(meth)acrylate,
5,7-bis[(2-butyloxetan-2-yl)propoxy]-1,3-adamantyl di(meth)acrylate,
5,7-bis[(2-methyloxetan-2-yl)butoxy]-1,3-adamantyl di(meth)acrylate,
5,7-bis[(2-ethyloxetan-2-yl)butoxy]-1,3-adamantyl di(meth)acrylate,
5,7-bis[(2-propyloxetan-2-yl)butoxy]-1,3-adamantyl di(meth)acrylate,
5,7-bis[(2-butyloxetan-2-yl)butoxy]-1,3-adamantyl di(meth)acrylate,
5,7-bis[(2-methyloxetan-2-yl)pentoxy]-1,3-adamantyl di(meth)acrylate,
5,7-bis[(2-ethyloxetan-2-yl)pentoxy]-1,3-adamantyl di(meth)acrylate,
5,7-bis[(2-propyloxetan-2-yl)pentoxy]-1,3-adamantyl di(meth)acrylate,
5,7-bis[(2-butyloxetan-2-yl)pentoxy]-1,3-adamantyl di(meth)acrylate,
5,7-bis[(2-methyloxetan-2-yl)hexoxy]-1,3-adamantyl di(meth)acrylate,
5,7-bis[(2-ethyloxetan-2-yl)hexoxy]-1,3-adamantyl di(meth)acrylate,
5,7-bis[(2-propyloxetan-2-yl)hexoxy]-1,3-adamantyl di(meth)acrylate,
5,7-bis[(2-butyloxetan-2-yl)hexoxy]-1,3-adamantyl di(meth)acrylate,
5,7-bis[(2-methyloxetan-2-yl)heptoxy]-1,3-adamantyl di(meth)acrylate,
5,7-bis[(2-ethyloxetan-2-yl)heptoxy]-1,3-adamantyl di(meth)acrylate,
5,7-bis[(2-propyloxetan-2-yl)heptoxy]-1,3-adamantyl di(meth)acrylate,
5,7-bis[(2-butyloxetan-2-yl)heptoxy]-1,3-adamantyl di(meth)acrylate,
7-[(3-methyloxetan-3-yl)methoxy]-1,3,5-adamantyl tri(meth)acrylate,
7-[(3-ethyloxetan-3-yl)methoxy]-1,3,5-adamantyl tri(meth)acrylate,
7-[(3-propyloxetan-3-yl)methoxy]-1,3,5-adamantyl tri(meth)acrylate,
7-[(3-butyloxetan-3-yl)methoxy]-1,3,5-adamantyl tri(meth)acrylate,
7-[(3-methyloxetan-3-yl)ethoxy]-1,3,5-adamantyl tri(meth)acrylate,
7-[(3-ethyloxetan-3-yl)ethoxy]-1,3,5-adamantyl tri(meth)acrylate,
7-[(3-propyloxetan-3-yl)ethoxy]-1,3,5-adamantyl tri(meth)acrylate,
7-[(3-butyloxetan-3-yl)ethoxy]-1,3,5-adamantyl tri(meth)acrylate,
7-[(3-methyloxetan-3-yl)propoxy]-1,3,5-adamantyl tri(meth)acrylate,
7-[(3-ethyloxetan-3-yl)propoxy]-1,3,5-adamantyl tri(meth)acrylate,
7-[(3-propyloxetan-3-yl)propoxy]-1,3,5-adamantyl tri(meth)acrylate,
7-[(3-butyloxetan-3-yl)propoxy]-1,3,5-adamantyl tri(meth)acrylate,
7-[(3-methyloxetan-3-yl)butoxy]-1,3,5-adamantyl tri(meth)acrylate,
7-[(3-ethyloxetan-3-yl)butoxy]-1,3,5-adamantyl tri(meth)acrylate,
7-[(3-propyloxetan-3-yl)butoxy]-1,3,5-adamantyl tri(meth)acrylate,
7-[(3-butyloxetan-3-yl)butoxy]-1,3,5-adamantyl tri(meth)acrylate,
7-[(3-methyloxetan-3-yl)pentoxy]-1,3,5-adamantyl tri(meth)acrylate,
7-[(3-ethyloxetan-3-yl)pentoxy]-1,3,5-adamantyl tri(meth)acrylate,
7-[(3-propyloxetan-3-yl)pentoxy]-1,3,5-adamantyl tri(meth)acrylate,
7-[(3-butyloxetan-3-yl)pentoxy]-1,3,5-adamantyl tri(meth)acrylate,
7-[(3-methyloxetan-3-yl)hexoxy]-1,3,5-adamantyl tri(meth)acrylate,
7-[(3-ethyloxetan-3-yl)hexoxy]-1,3,5-adamantyl tri(meth)acrylate,
7-[(3-propyloxetan-3-yl)hexoxy]-1,3,5-adamantyl tri(meth)acrylate,
7-[(3-butyloxetan-3-yl)hexoxy]-1,3,5-adamantyl tri(meth)acrylate,
7-[(3-methyloxetan-3-yl)heptoxy]-1,3,5-adamantyl tri(meth)acrylate,
7-[(3-ethyloxetan-3-yl)heptoxy]-1,3,5-adamantyl tri(meth)acrylate,
7-[(3-propyloxetan-3-yl)heptoxy]-1,3,5-adamantyl tri(meth)acrylate,
7-[(3-butyloxetan-3-yl)heptoxy]-1,3,5-adamantyl tri(meth)acrylate,
7-[(2-methyloxetan-2-yl)methoxy]-1,3,5-adamantyl tri(meth)acrylate,
7-[(2-ethyloxetan-2-yl)methoxy]-1,3,5-adamantyl tri(meth)acrylate,
7-[(2-propyloxetan-2-yl)methoxy]-1,3,5-adamantyl tri(meth)acrylate,
7-[(2-butyloxetan-2-yl)methoxy]-1,3,5-adamantyl tri(meth)acrylate,
7-[(2-methyloxetan-2-yl)ethoxy]-1,3,5-adamantyl tri(meth)acrylate,
7-[(2-ethyloxetan-2-yl)ethoxy]-1,3,5-adamantyl tri(meth)acrylate,
7-[(2-propyloxetan-2-yl)ethoxy]-1,3,5-adamantyl tri(meth)acrylate,
7-[(2-butyloxetan-2-yl)ethoxy]-1,3,5-adamantyl tri(meth)acrylate,
7-[(2-methyloxetan-2-yl)propoxy]-1,3,5-adamantyl tri(meth)acrylate,
7-[(2-ethyloxetan-2-yl)propoxy]-1,3,5-adamantyl tri(meth)acrylate,
7-[(2-propyloxetan-2-yl)propoxy]-1,3,5-adamantyl tri(meth)acrylate,
7-[(2-butyloxetan-2-yl)propoxy]-1,3,5-adamantyl tri(meth)acrylate,
7-[(2-methyloxetan-2-yl)butoxy]-1,3,5-adamantyl tri(meth)acrylate,
7-[(2-ethyloxetan-2-yl)butoxy]-1,3,5-adamantyl tri(meth)acrylate,
7-[(2-propyloxetan-2-yl)butoxy]-1,3,5-adamantyl tri(meth)acrylate,
7-[(2-butyloxetan-2-yl)butoxy]-1,3,5-adamantyl tri(meth)acrylate,
7-[(2-methyloxetan-2-yl)pentoxy]-1,3,5-adamantyl tri(meth)acrylate,
7-[(2-ethyloxetan-2-yl)pentoxy]-1,3,5-adamantyl tri(meth)acrylate,
7-[(2-propyloxetan-2-yl)pentoxy]-1,3,5-adamantyl tri(meth)acrylate,
7-[(2-butyloxetan-2-yl)pentoxy]-1,3,5-adamantyl tri(meth)acrylate,
7-[(2-methyloxetan-2-yl)hexoxy]-1,3,5-adamantyl tri(meth)acrylate,
7-[(2-ethyloxetan-2-yl)hexoxy]-1,3,5-adamantyl tri(meth)acrylate,
7-[(2-propyloxetan-2-yl)hexoxy]-1,3,5-adamantyl tri(meth)acrylate,
7-[(2-butyloxetan-2-yl)hexoxy]-1,3,5-adamantyl tri(meth)acrylate,
7-[(2-methyloxetan-2-yl)heptoxy]-1,3,5-adamantyl tri(meth)acrylate,
7-[(2-ethyloxetan-2-yl)heptoxy]-1,3,5-adamantyl tri(meth)acrylate,
7-[(2-propyloxetan-2-yl)heptoxy]-1,3,5-adamantyl tri(meth)acrylate,
7-[(2-butyloxetan-2-yl)heptoxy]-1,3,5-adamantyl tri(meth)acrylate,
3-[(oxirane-2-yl)methoxy]-1-adamantyl(meth)acrylate,
3-[(oxirane-2-yl)ethoxy]-1-adamantyl(meth)acrylate,
3-[(oxirane-2-yl)propoxy]-1-adamantyl(meth)acrylate,
3-[(oxirane-2-yl)butoxy]-1-adamantyl(meth)acrylate,
3-[(oxirane-2-yl)pentoxy]-1-adamantyl(meth)acrylate,
3-[(oxirane-2-yl)hexoxy]-1-adamantyl(meth)acrylate,
3,5-bis[(oxirane-2-yl)methoxy]-1-adamantyl(meth)acrylate,
3,5-bis[(oxirane-2-yl)ethoxy]-1-adamantyl(meth)acrylate,
3,5-bis[(oxirane-2-yl)propoxy]-1-adamantyl(meth)acrylate,
3,5-bis[(oxirane-2-yl)butoxy]-1-adamantyl(meth)acrylate,
3,5-bis[(oxirane-2-yl)pentoxy]-1-adamantyl(meth)acrylate,
3,5-bis[(oxirane-2-yl)hexoxy]-1-adamantyl(meth)acrylate,
3,5,7-tris[(oxirane-2-yl)methoxy]-1-adamantyl(meth)acrylate,
3,5,7-tris[(oxirane-2-yl)ethoxy]-1-adamantyl(meth)acrylate,
3,5,7-tris[(oxirane-2-yl)propoxy]-1-adamantyl(meth)acrylate,
3,5,7-tris[(oxirane-2-yl)butoxy]-1-adamantyl(meth)acrylate,
3,5,7-tris[(oxirane-2-yl)pentoxy]-1-adamantyl(meth)acrylate,
3,5,7-tris[(oxirane-2-yl)hexoxy]-1-adamantyl(meth)acrylate,
5-[(oxirane-2-yl)methoxy]-1,3-adamantyl di(meth)acrylate,
5-[(oxirane-2-yl)ethoxy]-1,3-adamantyl di(meth)acrylate,
5-[(oxirane-2-yl)propoxy]-1,3-adamantyl di(meth)acrylate,
5-[(oxirane-2-yl)butoxy]-1,3-adamantyl di(meth)acrylate,
5-[(oxirane-2-yl)pentoxy]-1,3-adamantyl di(meth)acrylate,
5-[(oxirane-2-yl)hexoxy]-1,3-adamantyl di(meth)acrylate,
5,7-bis[(oxirane-2-yl)methoxy]-1,3-adamantyl di(meth)acrylate,
5,7-bis[(oxirane-2-yl)ethoxy]-1,3-adamantyl di(meth)acrylate,
5,7-bis[(oxirane-2-yl)propoxy]-1,3-adamantyl di(meth)acrylate,
5,7-bis[(oxirane-2-yl)butoxy]-1,3-adamantyl di(meth)acrylate,
5,7-bis[(oxirane-2-yl)pentoxy]-1,3-adamantyl di(meth)acrylate,
5,7-bis[(oxirane-2-yl)hexoxy]-1,3-adamantyl di(meth)acrylate,
7-[(oxirane-2-yl)methoxy]-1,3,5-adamantyl tri(meth)acrylate,
7-[(oxirane-2-yl)ethoxy]-1,3,5-adamantyl tri(meth)acrylate,
7-[(oxirane-2-yl)propoxy]-1,3,5-adamantyl tri(meth)acrylate,
7-[(oxirane-2-yl)butoxy]-1,3,5-adamantyl tri(meth)acrylate,
7-[(oxirane-2-yl)pentoxy]-1,3,5-adamantyl tri(meth)acrylate,
7-[(oxirane-2-yl)hexoxy]-1,3,5-adamantyl tri(meth)acrylate.

Among the above-mentioned compounds, the adamantane derivatives represented by the general formulae (V), (VI), and (VII) are preferably used because the cured resins thereof are especially excellent in terms of transparency, heat resistance, and mechanical properties:

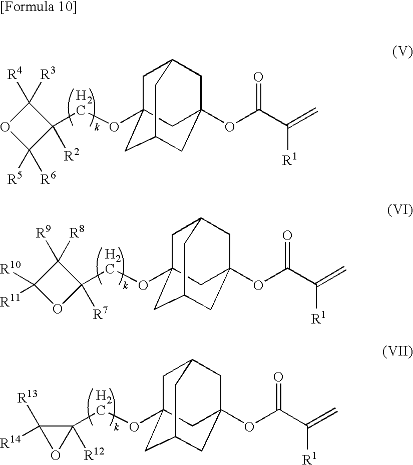

In the general formulae (V) to (VII), R¹ represents a hydrogen atom, an alkyl group having 1 to 4 carbon atoms, or a trifluoromethyl group; R² to R¹⁴ each independently represent a hydrogen atom, a fluorine atom, an alkyl group having 1 to 4 carbon atoms, a phenyl group, or a perfluoroalkyl group having 1 to 4 carbon atoms; k represents an integer of from 0 to 7.

Next, a preferable method for producing an adamantane derivative of the present invention will be described.

The adamantane derivative of the present invention can be synthesized by an etherification reaction of an alkanesulfonyloxyadamantyl acrylate with a cyclic ether-containing alcohol or by an etherification reaction of an alkanesulfonyloxyadamantyl acrylate with a halogen-containing alcohol, followed by a ring closure reaction.

The alkanesulfonyloxyadamantyl acrylate is a compound represented by the general formula (VIII):

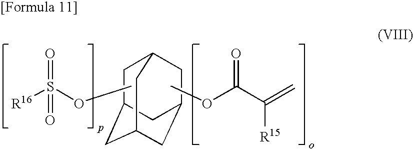

In the general formula (VIII), R¹⁵ represents a hydrogen atom, an alkyl group having 1 to 4 carbon atoms, or a trifluoromethyl group; R¹⁶ represents an alkyl group having 1 to 3 carbon atoms; o and p each independently represent an integer of 1 or larger, and o+p≦4.

Specific examples of an alkanesulfonyloxyadamantyl acrylate include
3-[(methylsulfonyl)oxy]-1-adamantyl meth(acrylate),
3,5-bis[(methylsulfonyl)oxy]-1-adamantyl meth(acrylate),
3,5,7-tris[(methylsulfonyl)oxy]-1-adamantyl meth(acrylate),
5-[(methyl sulfonyl)oxy]-1,3-adamantyl dimeth(acrylate),
5,7-bis[(methylsulfonyl)oxy]-1,3-adamantyl dimeth(acrylate),
7-[(methylsulfonyl)oxy]-1,3,5-adamantyl trimeth(acrylate), and the like.

The alkanesulfonyloxyadamantyl acrylates can be synthesized by heretofore known methods. For example, a method described in Japanese Patent Laid-Open Publication No. 2006-63061 may be used.

As the cyclic ether-containing alcohols, oxetanyl alcohols and oxiranyl alcohols may be mentioned.

The oxetanyl alcohols include (3-methyloxetan-3-yl)methanol, (3-ethyloxetan-3-yl)methanol, (3-propyloxetan-3-yl)methanol, (3-butyloxetan-3-yl)methanol, (3-methyloxetan-3-yl)ethanol, (3-ethyloxetan-3-yl)ethanol, (3-propyloxetan-3-yl)ethanol, (3-butyloxetan-3-yl)ethanol, (3-methyloxetan-3-yl)propanol, (3-ethyloxetan-3-yl)propanol, (3-propyloxetan-3-yl)propanol, (3-butyloxetan-3-yl)propanol, (3-methyloxetan-3-yl)butanol, (3-ethyloxetan-3-yl)butanol, (3-propyloxetan-3-yl)butanol, (3-butyloxetan-3-yl)butanol, (3-methyloxetan-3-yl)pentanol, (3-ethyloxetan-3-yl)pentanol, (3-propyloxetan-3-yl)pentanol, (3-butyloxetan-3-yl)pentanol, (3-methyloxetan-3-yl)hexanol, (3-ethyloxetan-3-yl)hexanol, (3-propyloxetan-3-yl)hexanol, (3-butyloxetan-3-yl)hexanol, (3-methyloxetan-3-yl)heptanol, (3-ethyloxetan-3-yl)heptanol, (3-propyloxetan-3-yl)heptanol, (3-butyloxetan-3-yl)heptanol, (2-methyloxetan-2-yl)methanol, (2-ethyloxetan-2-yl)methanol, (2-propyloxetan-2-yl)methanol, (2-butyloxetan-2-yl)methanol, (2-methyloxetan-2-yl)ethanol, (2-ethyloxetan-2-yl)ethanol, (2-propyloxetan-2-yl)ethanol, (2-butyloxetan-2-yl)ethanol, (2-methyloxetan-2-yl)propanol, (2-ethyloxetan-2-yl)propanol, (2-propyloxetan-2-yl)propanol, (2-butyloxetan-2-yl)propanol, (2-methyloxetan-2-yl)butanol, (2-ethyloxetan-2-yl)butanol, (2-propyloxetan-2-yl)butanol, (2-butyloxetan-2-yl)butanol, (2-methyloxetan-2-yl)pentanol, (2-ethyloxetan-2-yl)pentanol, (2-propyloxetan-2-yl)pentanol, (2-butyloxetan-2-yl)pentanol, (2-methyloxetan-2-yl)hexanol, (2-ethyloxetan-2-yl)hexanol, (2-propyloxetan-2-yl)hexanol, (2-butyloxetan-2-yl)hexanol, (2-methyloxetan-2-yl)heptanol, (2-ethyloxetan-2-yl)heptanol, (2-propyloxetan-2-yl)heptanol, (2-butyloxetan-2-yl)heptanol, and the like.

The oxiranyl alcohols include 3-epoxy-1-propanol, 4-epoxy-1-butanol, 5-epoxy-1-pentanol, 6-epoxy-1-hexanol, 7-epoxy-1-heptanol, and the like.

The halogen-containing alcohols used in the present invention generate cyclic ethers by a ring-closure reaction and, further, have reactivity with the alkanesulfonyloxyadamantyl acrylates. In the present invention, those which form 3-membered or 4-membered cyclic ethers are preferably used. A specific example of the ring-closure reaction includes a ring-closure reaction of a halohydrin compound. Also, as a group having reactivity with alkanesulfonyloxyadamantyl acrylates, a hydroxyl group is preferable.

Because of the above reason, polyhydric alcohols containing halogens are used preferably in the present invention.

The halogen-containing alcohols include 2-chloro-1,3-propanediol, 2-chloro-1,4-butanediol, 2-chloro-1,5-pentanediol, 2-chloro-1,6-hexanediol, 2-chloro-1,7-heptanediol, 2-chloro-1,8-octanediol, 2-bromo-1,3-propanediol, 2-bromo-1,4-butanediol, 2-bromo-1,5-pentanediol, 2-bromo-1,6-hexanediol, 2-bromo-1,7-heptanediol, 2-bromo-1,8-octanediol, 2-iodo-1,3-propanediol, 2-iodo-1,4-butanediol, 2-iodo-1,5-pentanediol, 2-iodo-1,6-hexanediol, 2-iodo-1,7-heptanediol, 2-iodo-1,8-octanediol, 3-chloro-1,2-propanediol, 4-chloro-1,3-butanediol, 5-chloro-1,4-pentanediol, 6-chloro-1,5-heptanediol, 7-chloro-1,6-hexanediol, 8-chloro-1,7-heptanediol, 9-chloro-1,8-octanediol, 3-bromo-1,2-propanediol, 4-bromo-1,3-butanediol, 5-bromo-1,4-pentadiol, 6-bromo-1,5-heptanediol, 7-bromo-1,6-hexanediol, 8-bromo-1,7-heptanediol, 9-bromo-1,8-octanediol, 3-iodo-1,2-propanediol, 4-iodo-1,3-butanediol, 5-iodo-1,4-pentanediol, 6-iodo-1,5-heptanediol, 7-iodo-1,6-hexanediol, 8-iodo-1,7-heptanediol, 9-iodo-1,8-octanediol, and the like.

In the above-mentioned etherification reaction, bases are generally used as catalysts and, if necessary, solvents are used. The bases include sodium amide, triethylamine, tributylamine, trioctylamine, pyridine, N,N-dimethylaniline, 1,5-diazabicyclo[4.3.0]nonene-5 (DBN), 1,8-diazabicyclo[5.4.0]undecene-7 (DBU), sodium hydroxide, potassium hydroxide, sodium hydride, potassium carbonate, silver oxide, sodium methoxide, potassium t-butoxide, sodium phosphate, sodium hydrogenphosphate, sodium dihydrogenphosphate, potassium phosphate, potassium hydrogenphosphate, potassium dihydrogenphosphate, and the like. These catalysts may be used by one kind singly or in a combination of two or more kinds.

As the solvents, used are those in which the alkanesulfonyloxyadamantyl acrylates have solubility of usually 0.5% by mass or more, preferably 5% by mass or more. The amount of the solvent used is such that the concentration of the alkanesulfonyloxyadamantyl acrylate in the reaction mixture is usually 0.5% by mass or more, preferably 5% by mass or more. In this case, the alkanesulfonyloxyadamantyl acrylate may be in a state of suspension but is preferably dissolved. Also, it is preferable to remove moisture contained in the solvent before use. Specifically, there may be cited hydrocarbon solvents such as hexane, heptane, and the like; ether solvents such as diethyl ether, 1,2-dimethoxyethane, tetrahydrofuran (THF), and the like; halogenated solvents such as dichloromethane, carbon tetrachloride, and the like; ester solvents such as ethyl acetate, butyl acetate, γ-butyrolactone, and the like; propylene glycol monomethyl ether acetate; dimethyl sulfoxide (DMSO); N,N-dimethyl formamide (DMF); and the like. These solvents may be used by one kind singly or as a mixture of two or more kinds.

As for the reaction temperature, usually, a range of from −200 to 200° C. is preferably used. Within this range, the reaction rate does not decrease and the reaction time does not become too long. Furthermore, production of a byproduct polymer does not increase. More preferably, the reaction temperature is in a range of from 50 to 150° C.

As for the reaction pressure, usually a range of from 0.01 to 10 MPa in absolute pressure is preferably used. Within this range, no particular high-pressure apparatus is necessary and is economic. More preferably, the pressure is from ordinary pressure to 10 MPa.

As for the reaction time, usually, a range of from 1 to 48 hours is preferably used.

When using the aforementioned halogen-containing alcohols as a raw material, in a case where production of the cyclic ether is insufficient, the desired compound is obtained by carrying out the ring-closure reaction under the following condition.

For the ring-closure reaction, a base catalyst is used. As the base catalysts, there may mentioned, for example, sodium hydroxide, potassium hydroxide, sodium phosphate, potassium phosphate, sodium carbonate, potassium carbonate, calcium hydroxide, and magnesium hydroxide. The amount of the base catalyst used is, relative to the adamantane derivative, usually 0.1 to 20% by mass, preferably 1 to 10% by mass. With the amount less than 0.1% by mass, the reaction time is prolonged and, with the amount exceeding 20% by mass, there is obtained little effect worth the addition of the catalyst.

As the solvent in the ring closure reaction, used are those in which the adamantane derivatives have solubility of usually 0.5% by mass or more, preferably 5% by mass or more. The amount of the solvent is such that the concentration of the adamantane derivative is usually 0.5% by mass or more, preferably 5% by mass or more. In this case, the adamantane derivative may be in a state of suspension but is preferably dissolved. Specifically, there may be mentioned hexane, heptane, toluene, DMF, N,N-dimethylacetamide, DMSO, ethyl acetate, diethyl ether, THF, acetone, methyl ethyl ketone, methyl isobutyl ketone, and the like. These solvents may be used by one kind singly or as a mixture of two or more kinds.

As for the reaction temperature, usually, a range of from 20 to 200° C. is preferably used. Within this range, the reaction rate does not decrease and the reaction time does not become too long. More preferably, the temperature is in a range of from 30 to 150° C.

As for the reaction pressure, usually a range of from 0.01 to 10 MPa in absolute pressure is preferably used. Within this range, no particular high-pressure apparatus is necessary and is economic. More preferably, the pressure is in a range of from ordinary pressure to 1 MPa.

As for the reaction time, it is usually from 1 minute to 24 hours, preferably from 30 minutes to 10 hours.

As for purification of the desired reaction product, there can be employed distillation, crystallization, column separation, and the like. The purification method may be selected in accordance with the properties of the product and the kinds of impurities.

In addition, identification of the compounds obtained can be carried out using gas chromatography (GC), liquid chromatography (LC), gas chromatography-mass spectrometry (GC-MS), nuclear magnetic resonance spectroscopy (NMR), infrared spectroscopy (IR), melting point measurement, and the like.

The resin composition of the present invention comprises an adamantane derivative represented by the general formula (I) as a resin, and, further, a polymerization initiator, and/or a curing agent. Because, as described above, the adamantane derivative of the present invention contains two different kinds of polymerizable groups, an acrylate portion and a cyclic ether portion, when producing a cured resin, the curing reaction may be chosen suitably in accordance with the purpose from various curing reactions including a radical polymerization reaction, a cationic polymerization reaction, and a reaction using a curing agent, and the like. Also, a cured resin may be obtained by combining a plurality of reactions, namely by combining a plurality of initiators and curing agents.

In carrying out a radical polymerization reaction, a radical polymerization initiator is used. In a case where curing is carried out by heat, a thermal polymerization initiator is selected and a photo-polymerization initiator will be selected when curing is carried out by light. The thermal polymerization initiators include organic peroxides such as benzoyl peroxide, methyl ethyl ketone peroxide, methyl isobutyl peroxide, cumene hydroperoxide, t-butyl hydroperoxide, and the like; and azo initiators such as azobisisobutyronitrile and the like. The photo-polymerization initiators include acetophenones, benzophenones, benzils, benzoin ethers, benzil diketals, thioxanthones, acylphosphine oxides, acylphosphine esters, aromatic diazonium salts, aromatic sulfonium salts, aromatic iodonium salts, aromatic iodosyl salts, aromatic sulfoxonium salts, metallocene compounds, and the like. The amount of the radical polymerization initiator added is, relative to the total amount of the composition, about 0.01 to 10% by weight, preferably 0.05 to 5% by weight, and these may be used singly or in combination.

In carrying out a cationic polymerization reaction, a cationic polymerization initiator is used, including a thermal- or photo-polymerization initiator which reacts with an oxetanyl group or oxiranyl group by heat or ultraviolet light. The cationic polymerization initiators include, for example, aromatic diazonium salts such as p-methoxybenzenediazonium hexafluorophosphate and the like; aromatic sulfonium salts such as triphenylsulfonium hexafluorophosphate and the like; aromatic iodonium salts such as diphenyliodonium hexafluorophosphate and the like; aromatic iodosyl salts; aromatic sulfoxonium salts; metallocene compounds; and the like. Among the above, the aromatic sulfonium salts such as triphenylsulfonium hexafluorophosphate and the aromatic iodonium salts such as diphenyliodonium hexafluorophosphate and the like are the most suitable. The amount of the cationic polymerization initiator added is, relative to the total amount of the composition, preferably 0.01 to 5.0% by mass, more preferably 0.1 to 3.0% by mass. With the amount of the initiator added in the range, excellent polymerization and physical properties such as optical characteristics can be realized. The polymerization initiators may be used singly or in a combination of two or more kinds.

In carrying out a curing reaction using a curing agent, there are used, as the curing agents, acid anhydride-based curing agents, phenol-based curing agents, amine-based curing agents, and the like.

The acid anhydride-based curing agents include phthalic acid anhydride, maleic acid anhydride, trimellitic acid anhydride, pyromellitic acid anhydride, hexahydrophthalic acid anhydride, tetrahydrophthalic acid anhydride, methylnadic acid anhydride, glutaric acid anhydride, methylhexahydrophthalic acid anhydride, methyltetrahydrophthalic acid anhydride, and the like.

The phenol-based curing agents include a phenol novolac resin, a cresol novolac resin, a bisphenol A novolac resin, a triazine-modified phenol novolac resin, and the like.

The amine-based curing agents include dicyandiamide, aromatic diamines such as m-phenylenediamine, 4,4′-diaminodiphenylmethane, 4,4′-diaminodiphenylsulfone, m-xylylenediamine, and the like.

The curing agents may be used singly or in a combination of two or more kinds. Among these curing agents, the acid anhydride curing agents are suitable in terms of physical properties such as transparency and the like of the cured resin. Above all, hexahydrophthalic acid anhydride, tetrahydrophthalic acid anhydride, methylhexahydrophtlalic acid anhydride, and methyltetrahydrophthalic acid anhydride are the most suitable. The amount of the curing agent is, relative to the total amount of the composition, preferably 0.01 to 10% by mass, more preferably 0.05 to 5% by mass. With the amount of the curing agent in the range, excellent polymerization and optical properties can be obtained.

In addition, in order to optimize the mechanical properties of the cured resin, and solubility and workability of the resin composition, the resin composition may contain resins other than the adamantane derivative which is represented by the general formula (I). As the other resins, epoxy resins are preferably used because they can improve the properties of the cured resin by making use of a cationic polymerization reaction or a reaction using a curing agent, which represent some aspects of the various reaction properties the adamantane derivative possesses.

The epoxy resins which may be used together with the adamantane derivatives represented by the general formula (I) include, for example, a bisphenol A epoxy resin, a bisphenol F epoxy resin, a bisphenol S epoxy resin (bisphenol A diglycidyl ether, bisphenol AD diglycidyl ether, bisphenol S diglycidyl ether, bisphenol F diglycidyl ether, bisphenol G diglycidyl ether, tetramethylbisphenol A diglycidyl ether, bisphenol hexafluoroacetone diglycidyl ether, bisphenol C diglycidyl ether, and the like); novolac epoxy resins such as a phenol novolac epoxy resin, a cresol novolac epoxy resin, and the like; an alicyclic epoxy resin; nitrogen-containing ring epoxy resins such as triglycidyl isocyanurate, a hydantoin epoxy resin, and the like; a hydrogenated bisphenol A epoxy resin; an aliphatic epoxy resin; a biphenyl epoxy resin which is the mainstream of low moisture-absorbing cured resin; a bicyclic epoxy resin, a naphthalene epoxy resin; polyfunctional epoxy resins such as trimethylolpropane polyglycidyl ether, glycerol polyglycidyl ether, and pentaerythritol polyglycidyl ether; a fluorine-containing epoxy resin such as a bisphenol AF epoxy resin, a (meth)acrylic acid glycidyl ether, and the like. These may be used singly or in a combination of two or more kinds.

The epoxy resins may either be solid or liquid at an ordinary temperature but, generally, the average epoxy equivalent of the epoxy resin used is preferably 100 to 2,000. When the epoxy equivalent is less than 100, there are cases where the hardened body of the epoxy resin composition becomes brittle. And, when the epoxy equivalent exceeds 2,000, there are cases where the glass transition temperature (Tg) of the hardened body becomes low.

Further, to the resin composition of the present invention, if necessary, there may be added suitably various additives, which have heretofore been used. The additives include, for example, a curing accelerator, an anti-deterioration agent, a modifying agent, a silane coupling agent, a defoaming agent, an inorganic filler, a solvent, a leveling agent, mold release agent, a dye, a pigment, and the like.

The curing accelerators are not particularly limited and include, for example, tertiary amines such as 1,8-diazabicyclo[5.4.0]undecene-7, triethylenediamine, 2,4,6-tris(dimethylaminomethyl)phenol, and the like; imidazoles such as 2-ethyl-4-methylimidazole, 2-methylimidazole, and the like; phosphorous compounds such as triphenylphosphine, tetraphenylphosphonium bromide, tetraphenylphosphonium tetraphenylborate, tetra-n-butylphosphonium o,o-diethyl phosphorodithioate and the like; quaternary ammonium salts; organometallic salts; the derivatives of these; and the like. These may be used singly or in a combination of two kinds or more. Among these curing accelerators, the tertiary amines, imidazoles, and phosphorous compounds are preferably used.

The content of the curing accelerator is, based on the total amount of the composition, preferably 0.01 to 8.0% by mass, more preferably 0.1 to 3.0% by mass. With the content of the curing accelerator within the range, a sufficient curing accelerating effect is obtained and the obtained cured resin does not show discoloration.

As the anti-deterioration agent, there may be mentioned heretofore known anti-deterioration agents such as, for example, phenol compounds, amine compounds, organic sulfur compounds, phosphorous compounds, and the like.

The phenol compounds include commercial products such as Irganox 1010 (Ciba Specialty Chemicals, trademark), Irganox 1076 (Ciba Specialty Chemicals, trademark), Irganox 1330 (Ciba Specialty Chemicals, trademark), Irganox 3114 (Ciba Specialty Chemicals, trademark), Irganox 3125 (Ciba Specialty Chemicals, trademark), Irganox 3790 (Ciba Specialty Chemicals, trademark), BHT, Cyanox 1790 (Cyanamid Co., trademark), Sumilizer GA-80 (Sumitomo Chemical Co., Ltd., trademark), and the like.

The amine compounds include Irgastab FS042 (manufactured by Ciba Specialty Chemicals, trademark); GENOX EP (manufactured by Crompton Corporation, trademark, chemical name; dialkyl-N-methylamine oxide), and the like. Further, there may be cited hindered amines such as ADK STAB LA-52, LA-57, LA-62, LA-63, LA-67, LA-68, LA-77, LA-82, LA-87 and LA-94 (manufactured by Adeka Corporation), Tinuvin 123, 144, 440 and 662, Chimassorb 2020, 119, and 944 (manufactured by CSC), Hostavin N30 (manufactured by Hoechst A.G.), Cyasorb UV-3346 and UV-3526 (manufactured by Cytec Industries Inc.), Uval 299 (manufactured by GLC Corporation), Sanduvor PR-31 (manufactured by Clariant Corporation), and the like.

As the organic sulfur compounds, there may be mentioned commercial products such as DSTP Yoshitomi (manufactured by Yoshitomiyakuhin Co., Ltd., trademark), DLTP Yoshitomi (manufactured by Yoshitomiyakuhin Co., Ltd., trademark), DLTOIB (manufactured by Yoshitomiyakuhin Co., Ltd., trademark), DMTP Yoshitomi (manufactured by Yoshitomiyakuhin Co., Ltd., trademark), Seenox 412S (manufactured by Shipro Kasei Kaisha, Ltd., trademark), Cyanox 1212 (manufactured by Cyanamid Corporation, trademark), and the like.

As the modifying agents, heretofore known modifying agents such as glycols, silicones, alcohols, and the like may be mentioned. As the silane coupling agents, heretofore known silane coupling agents, for example, silane-types, titanate-types, and the like may be cited. As the defoaming agents, heretofore known defoaming agents such as silicone-types and the like may be mentioned. As the inorganic powder, one having a particle size of from several nm to 10 μm may be used in accordance with the application and includes known inorganic powder such as glass powder, silica powder, titania, zinc oxide, alumina, and the like. As the solvents, which are used when the epoxy resin is in the form of powder or when a coating is diluted, there may be used aromatic solvents such as toluene, xylene, and the like, and ketone solvents such as methyl ethyl ketone, methyl isobutyl ketone, cyclohexanone, and the like.

As a method to cure the resin composition of the present invention, there may be used a method whereby the respective components of the above resin composition are mixed, poured into a mold (resin mold) or coated to obtain a desired shape, and, thereafter, cured by heating or irradiation of ultraviolet light. In the case of thermal curing, the curing temperature is 50 to 200° C., preferably 100 to 180° C. The curing temperature of 50° C. or higher can prevent occurrence of insufficient curing and a curing temperature of 200° C. or lower can prevent occurrence of coloration and the like. The curing time is preferably 0.5 to 6 hours, though it may vary with the resin composition. In the case of curing by irradiation of ultraviolet light, the irradiation intensity of the ultraviolet light is usually from about 500 to 5,000 mJ/cm², preferably from 1,000 to 4,000 mJ/cm². The ultraviolet irradiation may be followed by heating. In this case, heating is preferably carried out at 70 to 200° C. for 0.5 to 12 hours.

The molding method is not particularly limited and includes injection molding, blow molding, press molding, and the like, with injection molding being used preferably.

The cured resin obtained by curing the resin composition of the present invention has excellent heat resistance and transparency, with the total light transmittance being able to be 70% or more. Also, as is shown in the following Examples, there can be obtained a cured resin having a high glass transition temperature, excellent durability (heat resistance and light resistance), and excellent electrical properties such as dielectric constant and the like.

As described above, the cured resin of the present invention has excellent properties and can be used suitably in the field of electronic and optical materials. Specifically, it may be used preferably as resins (sealing material or adhesive agent) for optical semiconductors (LED and the like), flat panel displays (organic EL elements and the like), electronic circuits, and optical circuits (optical waveguide), and as optoelectronic members such as optical communication lenses, optical films, and the like.

### EXAMPLES

Next, the present invention will be described more specifically in terms of Examples. However, the present invention is not limited in anyway by these Examples. In the following Examples and Comparative Examples, evaluation of the obtained adamantane derivatives and cured resins were conducted as described below.

### (1) Glass Transition Temperature

A glass transition temperature was measured by using a solid viscoelasticity measuring instrument (manufactured by SII Nano Technology Inc., EXSTAR 6000 DMS) at a frequency of 1 Hz. The glass transition temperature, Tg, was obtained from the peak of the tan δ curve.

### (2) Flexural Strength

A flexural strength was measured according to JIS K6911.

### (3) Light Transmittance

A light transmittance was measured using a 3 mm thick specimen as a sample, according to JIS K7105 with a measurement wavelength of 400 nm (unit, %). As the measuring instrument, there was used a spectrophotometer, UV-3100S (manufactured by Shimadzu Corporation).

### (4) Nuclear Magnetic Resonance Spectrum

JNM-ECA 500 (manufactured by JEOL Ltd.) was used, with CDCl₃ employed as the solvent.

### (5) GC-MS (EI)

GCMS-QP 2010 (manufactured by Shimadzu Corporation) was used.

### Example 1

### Synthesis of 3-[(3-ethyloxetan-3-yl)methoxy]-1-adamantyl methacrylate

In a 500 ml 4-necked flask equipped with a reflux condenser, stirrer, thermometer, and nitrogen inlet tube were added 50.4 g (0.160 mol) of 3-methanesulfonyloxy-1-adamantyl methacrylate, 27.2 g (0.344 mol) of pyridine, 0.01 g of methoquinone, and 200 g of (3-ethyloxetan-3-yl)methanol (manufactured by Ube Industries, Ltd., trade name=Ethanacol EHO), and the atmosphere was replaced with nitrogen. Thereafter, the temperature of the reaction solution was raised to 120° C. and the solution was stirred for 4 hours under heating. After cooling the reaction solution, it was extracted with toluene and the extract washed with a saturated aqueous sodium chloride solution. The solvent was removed under reduced pressure to obtain 42.7 g (yield, 74%) of the desired product. The respective data of ¹H-NMR, ¹³C-NMR and GC-MS are shown below.

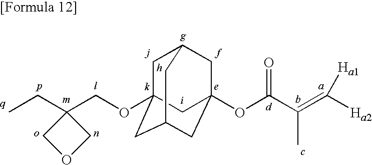

¹H-NMR (500 MHz): 0.85 (3H), 1 55 (2H), 1.67-1.78 (4H), 1.89 (2H), 2.06-2.20 (6H), 2.33 (2H), 3.53 (2H), 4.35-4.40 (4H), 5.48 (1H, a2), 6.00 (1H, a1).

¹³C-NMR (125 MHz): 8.25 (q), 18.39 (c), 26.64 (p), 31.06 (g), 35.26 (h), 40.35 (f or j), 40.51 (for j), 43.18 (m), 45.32 (i), 62.95 (k), 73.85 (e), 77.15 (l), 81.7 (n and o), 125.37 (a), 137.85 (b), 166.47 (d).

GC-MS (EI): 219 (27.68%), 151 (16.04%), 134 (19.91%), 115 (3.38%), 69 (100%).

### Example 2

### Synthesis of 3-[(3-ethyloxetan-3-yl)methoxy]-1-adamantyl acrylate

In a 500 ml 4-necked flask equipped with a reflux condenser, stirrer, thermometer, and nitrogen inlet tube were added 50.4 g (0.167 mol) of 3-methanesulfonyloxy-1-adamantyl acrylate, 27.2 g (0.344 mol) of pyridine, 0.01 g of methoquinone, and 200 g of (3-ethyloxetan-3-yl)methanol (manufactured by Ube Industries, Ltd., trade name=Ethanacol EHO), and the atmosphere was replaced with nitrogen. Thereafter, the temperature of the reaction solution was raised to 120° C. and the solution was stirred for 4 hours under heating. After cooling the reaction solution, it was extracted with toluene and the extract was washed with a saturated aqueous sodium chloride solution. The solvent was removed under reduced pressure to obtain 38 g (yield, 71.2%) of the desired product. The respective data of ¹H-NMR and ¹³C-NMR are shown below.

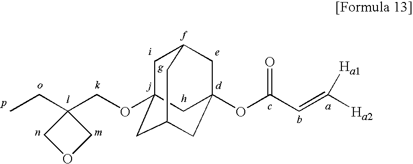

¹H-NMR (500 MHz): 0.85 (3H), 1.55 (2H), 1.67-1.78 (4H), 1.89 (2H), 2.06-2.20 (6H), 2.33 (2H), 3.53 (2H), 4.35-4.40 (4H), 5.69 (dd, J=1.6, 10.7 Hz, 1H, a2), 5.97 (dd, J=10.7, 17.6 Hz, 1H, b), 6.24 (dd, J=1.6, 17.6 Hz, 1H, a1).

¹³C-NMR (125 MHz): 8.25 (p), 26.64 (o), 30.96 (f), 35.04 (g), 40.20 (e or i), 40.39 (e or i), 43.18 (l), 45.19 (h), 62.95 (k), 74.67 (d or j), 81.51 (d or j), 81.7 (m and n), 129.87 (b), 130.11 (a), 165.25 (c).

### Example 3

### Synthesis of 3-[(oxiran-2-yl)methoxy]-1-adamantyl methacrylate

In a 500 ml 4-necked flask equipped with a reflux condenser, stirrer, thermometer, and nitrogen inlet tube were added 50.4 g (0.160 mol) of 3-methanesulfonyloxy-1-adamantyl methacrylate, 27.2 g (0.344 mol) of pyridine, 0.01 g of methoquinone, and 200 g of 2-chloro-1,3-propanediol, and the atmosphere was replaced with nitrogen. Thereafter, the temperature of the reaction solution was raised to 80° C. and the solution was stirred for 2 hours under heating. After cooling the reaction solution, it was extracted with 500 ml of toluene and the extract was washed twice with 500 ml of a saturated aqueous sodium chloride solution. To the solution after washing was added 20 g of sodium hydroxide and the mixture was stirred under heating at 110° C. for 2 hours, followed by cooling of the solvent. After washing twice with 300 ml of a saturated aqueous sodium chloride solution, the solvent was removed under reduced pressure to obtain 40 g (yield, 82%) of the desired product. The respective data of ¹H-NMR and ¹³C-NMR are shown below.

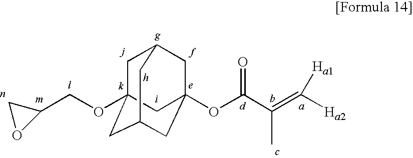

¹H-NMR (500 MHz): 1.67-1.78 (4H), 1.89 (2H), 2.06-2.20 (6H), 2.33 (2H), 2.38 (1H), 2.63 (1H), 2.86 (1H), 3.53 (2H), 5.48 (1H, a2), 6.00 (1H, a1).

¹³C-NMR (125 MHz): 18.39 (c), 31.06 (g), 35.26 (h), 40.35 (f or j), 40.51 (for j), 44.2 (n), 51.0 (m), 45.32 (i), 62.95 (k), 69.4 (l), 73.85 (e), 125.37 (a), 137.85 (b), 166.47 (d).

### Production of Cured Resin

### Examples 4 to 6, Comparative Examples 1 and 2

The respective components were mixed in a composition and amounts (parts by mass) shown in Table 1 and deaerated to obtain a resin composition. Subsequently, a cured resin (1 mm thick sheet) was produced by heating at 70° C. for 4 hours, at 110° C. for 2 hours, and then at 150° C. for 2 hours. The cured resin obtained was subjected to various tests. The evaluation results are shown in Table 2.

TABLE 1

| | | | | | Comparative | |
|---|---|---|---|---|---|---|
| | | Example | | | Example | |
| | | 4 | 5 | 6 | 1 | 2 |
| Resin | Adamantane | 100 | — | 80 | — | — |
| | derivative | | | | | |
| | (Example 1) | | | | | |
| | Adamantane | — | 100 | — | — | — |
| | derivative | | | | | |
| | (Example 3) | | | | | |
| | Adamantyl | — | — | — | 100 | — |
| | methacrylate | | | | | |
| | Glycidyl | — | — | — | — | 100 |
| | methacrylate | | | | | |
| | Alicyclic epoxy | — | — | 20 | — | — |
| | resin | | | | | |
| Polymerization | Aromatic sulfonium | — | — | 2 | — | — |
| initiator | salt | | | | | |
| | Organic peroxide | 0.5 | 0.5 | 0.5 | 0.5 | 0.5 |

Alicyclic epoxy resin: manufactured by Daicel Cytec Co., Ltd.; trade name, CELLOXIDE 2021P
Aromatic sulfonium salt: manufactured by Sanshin Chemical Industry Co., Ltd.; trade name, San-Aid SI-100L
Organic peroxide: manufactured by NOF Corporation; trade name, PEROYL TCP

TABLE 2

| | | | | Comparative | |
|---|---|---|---|---|---|
| | Example | | | Example | |
| | 4 | 5 | 6 | 1 | 2 |
| Glass transition temperature (° C.) | 150 | 151 | 170 | 170 | — |
| Flexural strength (MPa) | 20 | 19 | 60 | 10 | — |
| Light transmittance (%) | 92 | 90 | 89 | 92 | 91 |

The adamantane derivative of the present invention comprises two kinds of polymerizable groups. Therefore, it is possible to cure the same by either a cationic polymerization reaction or a radical polymerization reaction, and it can be used under various curing conditions in accordance with various intended uses of the cured resin.

In addition, as is shown in Example 6, it is possible to improve the desired properties considerably, applying a wide range of reactivity the adamantane derivative possesses, by using the adamantane derivative in combination with other resins.

The above effect was attained without degrading the properties as a polymerizable monomer. Namely, as is shown in Examples 4 and 5, and Comparative Examples 1 and 2, the cured resin obtained by use of the adamantane derivative of the present invention has, in comparison with conventional high-performance resins, equivalent performances (heat resistance and transparency) or a more excellent performance (flexural strength).

### INDUSTRIAL APPLICABILITY

According to the present invention, (1) there is provided a cured resin which has excellent optical properties such as transparency and (long-term) light stability, long-term heat resistance, dielectric constant, and mechanical properties, and can be utilized suitably in the field of electronic and optical materials and, further, (2) there is provided an adamantane derivative which can be used for the production of the cured resin and to which can be applied numerous ways of use depending on a variety of intended uses of the cured resin.

1. An adamantane derivative having a structure represented by the general formula (I): 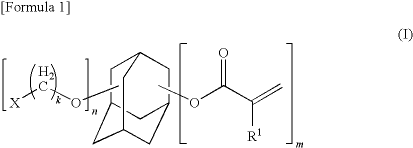
wherein R¹ represents a hydrogen atom, an alkyl group having 1 to 4 carbon atoms, or a trifluoromethyl group; X represents a cyclic ether group which can undergo cationic ring-opening polymerization; k represents an integer of from 0 to 7; m and n each independently represent an integer of 1 or larger, and m+n≦4.

2. The adamantane derivative according to claim 1, wherein X in the general formula (I) is a group represented by the general formula (II) or (III): 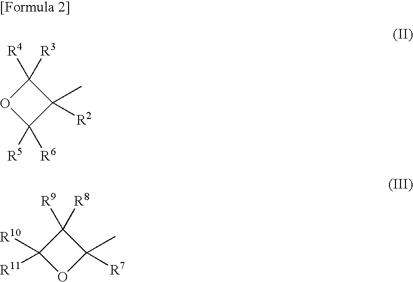
wherein R² to R¹¹ each independently represent a hydrogen atom, a fluorine atom, an alkyl group having 1 to 4 carbon atoms, a phenyl group, or a perfluoroalkyl group having 1 to 4 carbon atoms.

3. The adamantane derivative according to claim 1, wherein X in the general formula (I) is a group represented by the general formula (IV): 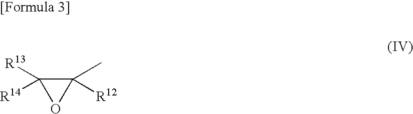
wherein R¹² to R¹⁴ each independently represent a hydrogen atom, a fluorine atom, an alkyl group having 1 to 4 carbon atoms, a phenyl group, or a perfluoroalkyl group having 1 to 4 carbon atoms.

4. An adamantane derivative represented by the general formula (V) or (VI): 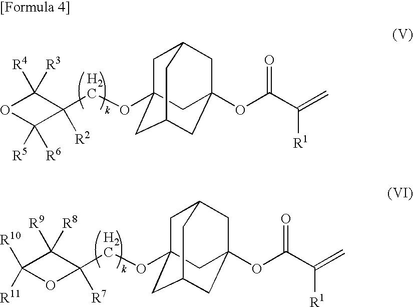
wherein R¹ represents a hydrogen atom, an alkyl group having 1 to 4 carbon atoms, or a trifluoromethyl group; R² to R¹¹ each independently represent a hydrogen atom, a fluorine atom, an alkyl group having 1 to 4 carbon atoms, a phenyl group, or a perfluoroalkyl group having 1 to 4 carbon atoms; k represents an integer of from 0 to 7.

5. An adamantane derivative represented by the general formula (VII): 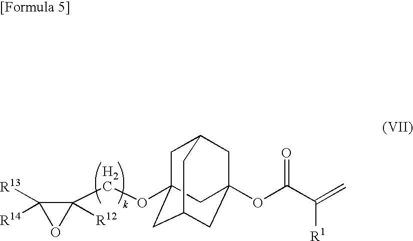
wherein R¹ represents a hydrogen atom, an alkyl group having 1 to 4 carbon atoms, or a trifluoromethyl group; R¹² to R¹⁴ each independently represent a hydrogen atom, a fluorine atom, an alkyl group having 1 to 4 carbon atoms, a phenyl group, or a perfluoroalkyl group having 1 to 4 carbon atoms; k represents an integer of from 0 to 7.

6. A method for producing the adamantane derivative according to claim 2, wherein oxetanyl alcohol and an adamantane derivative represented by the general formula (VIII) are reacted: 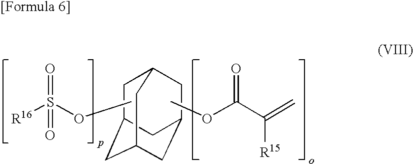
wherein R¹⁵ represents a hydrogen atom, an alkyl group having 1 to 4 carbon atoms, or a trifluoromethyl group; R¹⁶ represents an alkyl group having 1 to 3 carbon atoms; o and p each independently represent an integer of 1 or larger, and o+p≦4.

7. A method for producing an adamantane derivative according to claim 3, wherein a halogen-containing alcohol and an adamantane derivative represented by the general formula (VIII) are reacted.

8. A cured resin obtained by polymerizing the adamantane derivative represented by the general formula (I) by a cationic polymerization initiator and/or a radical polymerization initiator.
